(19) United States
(12) Patent Application Publication
(10) Pub. No.: US 2021/0239576 A1
(43) Pub. Date: Aug. 5, 2021

(54) DETECTOR FOR DETECTING A COMPLEX STATE OF AN OBJECT, ELECTRONIC EAR AND DETECTING METHOD

(71) Applicant: Orange, Paris (FR)

(72) Inventors: Julien Riera, CHÂTILLON CEDEX (FR); Nicolas Pellen, CHÂTILLON CEDEX (FR); Rozenn Nicol, CHÂTILLON CEDEX (FR); Katell Peron, CHÂTILLON CEDEX (FR)

(21) Appl. No.: 17/049,904

(22) PCT Filed: Apr. 25, 2019
(86) PCT No.: PCT/FR2019/050975

§ 371 Date: Oct. 22, 2020

(30) Foreign Application Priority Data
Apr. 26, 2018 (FR) 1853673

(51) Int. Cl. G01M 99/00; G10L 25/51; H04R 1/08

(52) U.S. Cl. CPC G01M 99/005; A61B 7/04; H04R 1/08; G10L 25/51

(57) ABSTRACT

Methods are described of monitoring or supervising objects, particularly non-communicating objects. A detector for detecting complex states of objects is described, the detector comprising an analyzer configured to determine an object-related complex state depending on recognition of a least one sound emitted by the object. The recognized sound allows the detector to determine the complex state of an object. This complex state of the object may include an abnormal operating state of the object or a state of the object that is not related to its operation. For example, the complex state of the object may include its position on a surface, in a volume, or the identity of the person having handled the object.

The invention relates to the monitoring, even the supervision, of objects, in particular of non-communicating objects.

In order to supervise different objects in our everyday life, the promise was made to make the home smart, and more generally make the various living and working spaces smart. In particular, the aim of home automation is to centralize the control of different electronic systems (heating, alarm, etc.) in the home, hotels, school, etc., to meet the human needs of comfort, safety, etc. Nevertheless, while the smart home has been able to meet some needs by controlling the heating, the openings (doors, windows, blinds), the alarm, it has not been rolled out across the board because it is unattractive because of the installation costs.

With the development of the Internet of Things, the monitoring of objects remotely is now possible provided that the objects are connected, even smart. Supervising an object requires the home to be equipped with connected and/or smart objects: dishwasher, refrigerator, electrical power outlet, etc., connected detectors (door opening, camera, etc.). This can prove costly because of the large number of smart objects required to equip the home and the individual additional cost of smart equipment as opposed to unconnected equipment.

The connected detectors can provide a response to a simple question by detecting a simple, often binary, state: active/inactive, open/closed, etc., of an object. A simple state can be defined as a state of normal operation of an object and/or of an element of an object: active/inactive, open/closed, operating mode. For example, for a washing machine, the detector is capable of indicating the simple states of the washing machine: door open/closed, washing in progress or stopped, type of wash. The current connected detectors do not make it possible to address complex questions such as: where are my keys? Why is my appliance no longer operating? How can I restore it to operation?

One of the aims of the present invention is to provide enhancements compared to the state of the art.

One subject of the invention is a detector of complex states relating to an object comprising an analyzer capable of determining a complex state relating to the object as a function of a recognition of at least one sound emitted by the object. Thus, the recognized sound allows the detector to determine the complex state of an object, that is to say, notably, an abnormal state of operation of the object or a state of the object which does not relate to its operation: for example its position on a surface, within a volume, the identity of the person having manipulated the object, etc.

Advantageously, the complex state of the object is a complex state from among the following: a state of anomalous operation of the object, a relative position of the object, an identity of the manipulator of the object. Thus, the detector makes it possible to warn of a malfunction of the object even if the object is not smart, and/or inform of a passive parameter relating to an object even if it is not smart, even for passive objects: position in a closed or open volume (room, apartment, home, garden, etc.), identity of the manipulator, etc.

Advantageously, the analyzer is capable of determining a complex state of the object as a function of the context of recognition of the sound emitted by the object. Thus, the analyzer is capable of distinguishing two complex states corresponding to one and the same recognized sound. For example, a noise from the dishwasher can correspond to two states of the dishwasher: normal, anomaly. In this case, the context will allow the analyzer to determine which of the two states: normal, anomaly, is the current state of the object. For example, in a context of activity of the dishwasher, the recognized sound will correspond to a normal state of operation whereas, in a context of stoppage of the dishwasher, the same recognized sound will correspond to a state of anomalous operation.

Advantageously, the complex anomalous state is a type of anomalous operation of the object. Thus, the detector makes it possible not only to warn of a malfunction but, in addition, of the type of malfunction allowing the user or a supervising device to correct, possibly, the malfunction of the object.

Advantageously, the detector is a connected detector. Thus, the detector can communicate the detected complex state to a third-party device, notably a non-smart object supervisor making it possible to trigger an intervention of the user of the object, of a technician, or a processing of a supervisor device, notably to transmit a message that is a function of the complex state to a communication device of a user, of a technician and/or correct the malfunction when the complex state is an operating anomaly.

Another subject of the invention is an electronic ear comprising a first analyzer capable of recognizing at least one sound emitted by an object and a second analyzer capable of diagnosing the complex state of the object as a function of at least one recognized sound.

Advantageously, the electronic ear comprises a sound sensor capable of capturing at least one sound emitted by the object. Thus, the errors associated with the degradation of the sound upon the transmission between the sensor and the first analyzer performing the sound recognition are eliminated.

Advantageously, the electronic ear comprises an object supervisor capable of determining at least one processing to be executed as a function of the diagnosed complex state. Thus, the electronic ear makes it possible, by virtue of said object supervisor device, not only to diagnose a complex state, notably an anomaly, but also to trigger an action as a function of this complex state: transmission of a message that is a function of the diagnosed complex state, correction of the diagnosed anomaly either by a human being or by the supervisor of the electronic ear (which notably controls either the object if it is smart, or a third-party device), or by a remote supervisor.

Advantageously, the electronic ear comprises a communication interface capable of establishing a communication on command from the supervisor, when the processing to be executed comprises a connection with a remote device. Thus, the electronic ear makes it possible not only to diagnose an anomaly but also to trigger its remote management: by a remote supervisor device, a user, a technician, etc.

Advantageously, the electronic ear comprises fixing agents and is intended to be fixed onto the object emitting the recognized sound, the sensor is position in the part of the electronic ear close to the fixing agents and/or oriented in the electronic ear toward the plane formed by the fixing agents. Thus, the sound picked up comprises less noise, that is to say sound originating from the environment of the object, and more sound originating from the object itself, allowing for an improvement of the recognition by the first analyzer.

Another subject of the invention is a method for detecting complex states relating to an object comprising a diagnosis determining a complex state relating to the object as a function of a recognition of at least one sound emitted by the object.

Another subject of the invention is also a method for monitoring objects comprising a recognition of at least one sound emitted by an object and a diagnosis of the complex state of the object as a function of at least one recognized sound.

Another subject of the invention is also a method for supervising objects comprising a determination of a processing to be executed as a function of the complex state diagnosed as a function of a recognition of at least one sound emitted by the object.

Advantageously, the supervision method comprises a triggering of the execution of the determined processing.

Advantageously, according to one implementation of the invention, the different steps of the method according to the invention are implemented by software or a computer program, this software comprising software instructions intended to be executed by a data processor of a device forming part of a detector of complex states of an object and/or of an electronic ear and being designed to control the execution of the different steps of this method. The invention therefore also targets a program comprising program code instructions for executing the steps of the method for detecting anomalies and/or the method for monitoring objects when said program is run by a processor. This program can use any programming language and be in the form of source code, object code, or intermediate code between source code and object code, such as in a partially compiled form or in any other desirable form.

The features and advantages of the invention will become more clearly apparent on reading the description, given by way of example, and the figures relating thereto which represent:

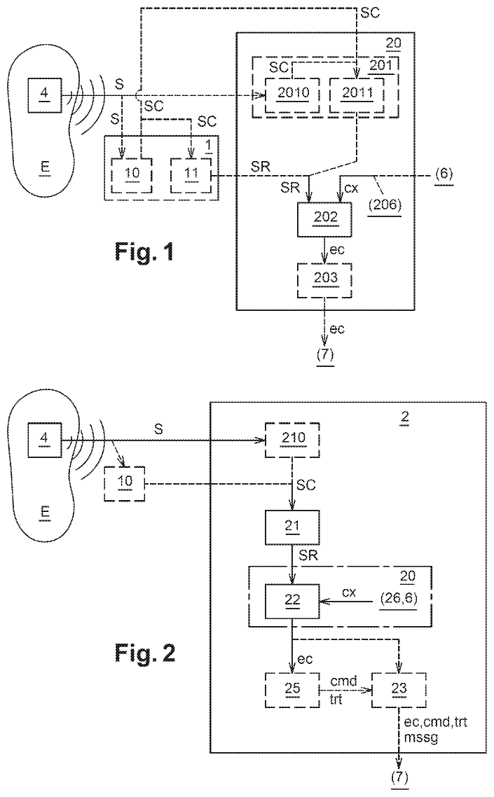

FIG. 1, a simplified diagram of a detector of complex states according to the invention,

FIG. 2, a simplified diagram of an electronic ear according to the invention,

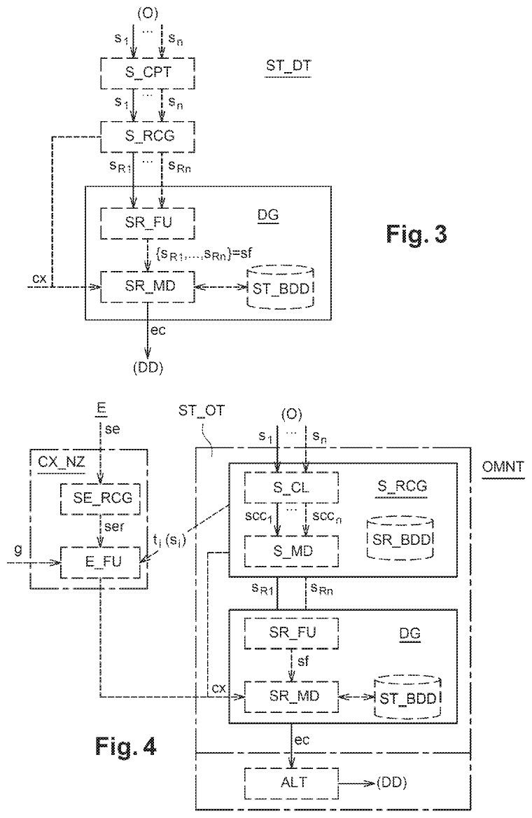

FIG. 3, a simplified diagram of the method for detecting complex states according to the invention,

FIG. 4, a simplified diagram of the method for monitoring objects according to the invention,

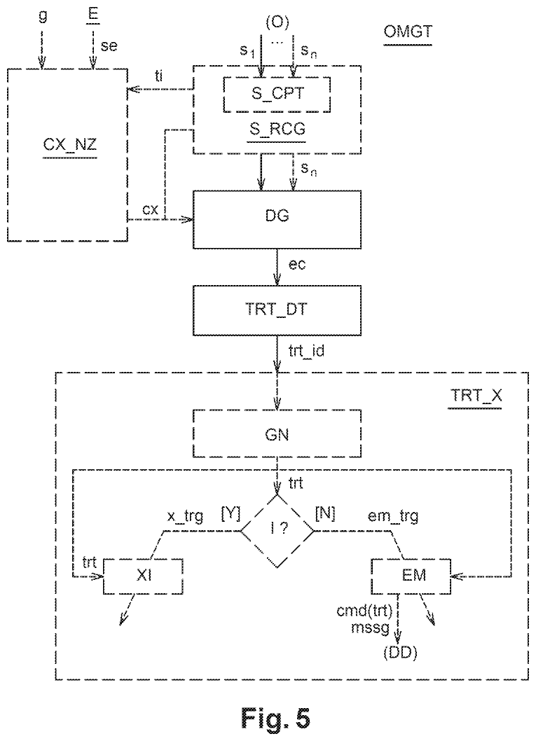

FIG. 5, a simplified diagram of the method for supervising objects according to the invention,

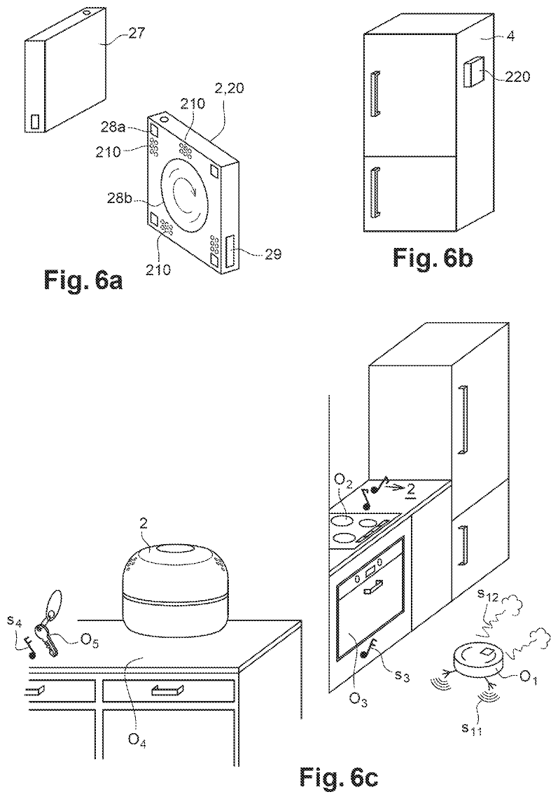

FIGS. 6a to 6c, diagrams of cases of use of the invention, respectively a detector or an electronic ear that can be fixed onto an object, said detector or ear positioned on a refrigerator constituting the object, a multi-object detector or electronic ear.

Complex states are understood to mean the non-functional states of an object as opposed to the simple, generally binary, states which describe a state of operation of the object: active/inactive, open/closed, mode of operation. In particular, the complex states, also called non-functional states, comprise the states of anomalous operation of the object, a relative position of the object, an identity of the manipulator of the object.

An object is understood to mean a concrete thing that can be touched and which is assigned a precise use. For example, an object can be a domestic object such as a cabinet, but also a domestic electric appliance, an element of the home: door, window, blind, etc.

The context of recognition of a sound is understood to mean all the circumstances surrounding the recognition of the sound, such as one or more simple and/or complex states of one or more objects, the instant of recognition of the sound.

FIG. 1 illustrates a simplified diagram of a detector of complex states according to the invention. The method for detecting ST_DT complexes relating to an object 4 comprises an analyzer 202 capable of determining a complex state ec relating to the object as a function of a recognition of at least one sound s emitted by the object 4. Thus, the recognized sound sr allows the detector 20 to determine the complex state ec of an object 4, that is to say, notably, a state of abnormal operation of the object 4 or a state of the object 4 which does not relate to its operation: for example its position on a surface, within a volume, the identity of the person having manipulated the object, etc.

In particular, the complex state ec of the object 4 is a complex state from among the following: a state of anomalous operation of the object, a relative position of the object, an identity of the manipulator of the object. Thus, the detector 20 makes it possible to warn of a malfunction of the object 4 even if the object 4 is not smart, and/or inform of a passive parameter relating to an object 4 even if it is not smart, even for passive objects 4: position in a closed or open volume (room, apartment, home, garden, etc.), identity of the manipulator, etc. A passive parameter is a parameter that is different from an active parameter or an activity parameter, that is to say a parameter relating to the operation of the object 4.

In particular, the analyzer 202 is capable of determining a complex state ec of the object 4 as a function of the context cx of recognition of the sound s emitted by the object 4. The recognition of the sound s is notably implemented by a first analyzer 11, 2011 (the analyzer 202 then being called second analyzer). Thus, the analyzer 202 or second analyzer is capable of distinguishing two complex states corresponding to one and the same recognized sound sr according to the context cx. For example, a noise from the dishwasher can correspond to two states of the dishwasher: normal or anomaly. In this case, the context cx will allow the analyzer 202 to determine which of the two states, normal or anomaly, is the current state of the object 4. For example, in a context cx of activity of the dishwasher 4, the recognized sound sr will correspond to a normal state of operation (simple state) whereas, in a context cx of stoppage of the dishwasher 4, the same recognized sound sr will correspond to a state of anomalous operation (complex state ec).

The natural or manufactured objects in our environment make noise, in their operation (example: engine of a boat), in their uses (example: the click of a ballpoint pen or the noise of a closing door), or upon events (example: keys that are placed on a table). Some manufactured objects produce sounds in their operation so that the human being recognizes them and can thus deduce information on the state of the object (example: at the end-of-cycle sound of a washing machine).

In particular, the complex anomalous state ec is a type of anomalous operation of the object. Thus, the detector 20 makes it possible not only to warn of a malfunction but, in addition, of the type of malfunction, allowing a user (notably a user of the object 4) or a supervisor device 7, to correct, possibly, the malfunction of the object 4.

In particular, the detector 20 is a connected detector. Thus, the detector 20 can communicate the detected complex state ec to a third-party device 7, notably a non-smart object supervisor making it possible to trigger an intervention from the user of the object, from a technician, or a processing of a supervisor device, notably to transmit a message that is a function of the complex state to a communication device of a user, of a technician and/or correct the malfunction when the complex state is an operating anomaly.

The sound s emitted by the object 4 is picked up either by a sensor 10 external to the detector 20, or a sensor 2010 implemented in the detector 20. A sound is understood to mean not only the sounds that are audible to the human being but also all other vibrations perceived by a living being or a machine, therefore also infrasound, ultrasound, but also mechanical vibrations.

The sound picked up sc is supplied to a first analyzer 11, 2011 capable of recognizing sounds, that is to say of identifying the movement, the friction, etc., of the object 4 originating the sound (closure of a door, placement of a key on a table, noise from an electric motor, etc.). This first sound recognition analyzer 11, 2011 is either a first analyzer 11 external to the detector 20, or a first analyzer 2011 implemented in the detector 20. Possibly, the sensor 10, 2010 and the first analyzer 11, 2011 are implemented in one and the same sound processing device 1, 201 which is either a processing device 1 external to the detector 20, or a processing device 201 implemented in the detector 20.

In particular, a third analyzer 6, 206, called context analyzer, supplies, to the second analyzer 202 called complex state analyzer, the context cx in which the sound sr was recognized. The context analyzer 6, 206 is either an analyzer 6 external to the detector 20, or an analyzer 206 implemented in the detector 20. The context analyzer receives, notably, information t(s) relating to the moment of capture of the sound s for example from a clock or a time stamper of the sensor 10, 2010, information g relating to the position of the object 4 upon the emission of the sound s picked up, and other information relating to the environment of the object 4, in particular information relating to the sound environment se of the object 4 upon the capture of the sound s emitted (supplied by the sensor 10, 2010 or one of other sensors present in the environment E of the object 4) or information relating to the preceding states ep of the object 4 (simple and/or complex states ec).

In particular, the detector 20 comprises a transmitter 203 capable of transmitting to a third-party device 7. The transmitter 203 can transmit either the detected complex state ec, or a message comprising or that is a function of the detected complex state mssg(ec), etc. Thus, the third-party device 7 can implement a processing as a function of the complex state ec received or simply display/reproduce this information to the user of the third-party device 7. The third-party device 7 can be a simple display screen, a smartphone, a computer of the user of the object or of a third-party user (parent, technician, etc.), a server or device providing a service in an Internet network, etc.

FIG. 2 illustrates a simplified diagram of an electronic ear according to the invention. The electronic ear 2 comprises a first analyzer 21 capable of recognizing at least one sound s emitted by an object 4 and a second analyzer 22 capable of diagnosing the complex state ec of the object 4 as a function of at least one recognized sound sr. Thus, the recognized sound sr allows the electronic ear 2 to determine the complex state ec of an object 4, that is to say, notably, a state of abnormal operation of the object 4 or a state of the object 4 which does not relate to its operation: for example its position on a surface, in a volume, the identity of the person having manipulated the object, etc.

In particular, the complex state ec of the object 4 is a complex state from among the following: a state of anomaly relating to the object, a relative position of the object, an identity of the manipulator of the object, etc. Thus, the electronic ear 2 makes it possible to warn of a malfunction of the object 4 even if the object 4 is not smart, and/or inform of a passive parameter relating to an object 4 even if it is not smart, even for passive objects 4: position in a closed or open volume (room, apartment, home, garden, etc.), identity of the manipulator, etc. A passive parameter is a parameter that is different from an active parameter or activity parameter, that is to say a parameter relating to the operation of the object 4.

In particular, the complex anomalous state ec is a type of anomaly relating to the object. Thus, the electronic ear 2 makes it possible not only to warn of a malfunction but, in addition, of the type of malfunction allowing a user (notably a user of the object 4) or a supervisor device 7, to correct, possibly, the malfunction of the object 4.

In particular, the second analyzer 22 is capable of diagnosing a complex state ec of the object as a function of the context cx of recognition of the sound s emitted by the object 4. Thus, for the same recognized sound, the diagnosed complex state ec will not be the same according to the context cx. For example, in the context cx of an appliance 4 with the door open, a sound s from the appliance 4 will correspond to a normal state of operation (simple state), whereas, in a context cx of the appliance 4 with the door closed, this same sound s will be diagnosed as corresponding to an anomalous state (complex state ec).

In particular, the electronic ear 2 comprises a sound sensor 210 capable of capturing at least one sound s emitted by the object 4. Thus, the errors linked to the degradation of the sound upon the transmission between the sensor 210 and the first analyzer 21 performing the sound recognition are eliminated.

In particular, the electronic ear 2 comprises an object supervisor 25 capable of determining at least one processing trt to be executed as a function of the diagnosed complex state ec. Thus, the electronic ear 2 makes it possible, by virtue of said object supervisor 25 device, not only to diagnose a complex state ec, notably an anomaly, but also to trigger an action trt as a function of this complex state: transmission of a message mssg that is a function of the diagnosed complex state, correction of the diagnosed anomaly either by a human being or by the supervisor of the electronic ear (which notably controls either the object if it is smart, or a third-party device), or by a remote supervisor.

In particular, the electronic ear 2 comprises a communication interface 23 capable of establishing a communication on command cmd from the supervisor 25, when the processing to be executed comprises a connection with a remote device 7. The communication interface or transmitter 23 can, notably, transmit either the detected complex state ec, or a message comprising or that is a function of the detected complex state mssg(ec), etc. Thus, the third-party device 7 can implement a processing as a function of the complex state ec received or simply display/reproduce this information to the user of the third-party device 7. The third-party device 7 can be a simple display screen, a smartphone, a computer of the user of the object or of a third-party user (parent, technician, etc.), a server or device providing a service in an Internet network, etc. Thus, the electronic ear 2 makes it possibly not only to diagnose an anomaly but also to trigger its remote management: by a remote supervisor device, a user, a technician, etc.

In particular, the electronic ear 2 comprises fixing agents 28 capable of allowing the electronic ear 2 to be fixed at least temporarily onto an object: the object 4 or another object of the environment E of the object 4. Thus, the electronic ear 2 makes it possible to detect the complex states of several objects 4 present in the same environment E. Also, the recognition of the first analyzer 21 and/or the diagnosis of the second analyzer 22 are not disturbed by a movement of the electronic ear 2.

In particular, the electronic ear 2 comprises fixing agents 28 (illustrated in the FIG. 6a) and is intended to be fixed onto the object 4 emitting the recognized sound s. The sensor 210 is positioned in the part of the electronic ear 2 close to the fixing agents 28 and/or oriented in the electronic ear 2 toward the plane formed by the fixing agents 28. Thus, the sound s picked up comprises less noise, that is to say sound originating:
from a movement of the electronic ear 2 and/or
from the environment E of the object 4, and

more sound originating from the object 4 itself, allowing for an improvement of the recognition by the first analyzer 21.

Notably, the electronic ear comprises a method for detecting ST_DT complexes relating to an object 4 implementing the second analyzer 202 capable of determining a complex state ec relating to the object as a function of a recognition of at least one sound s emitted by the object 4.

In particular, the electronic ear 2 is a connected electronic ear. Thus, the diagnosed complex states are transmitted to the Internet network notably to be stored and to retain a history of the object 4.

Furthermore, the electronic ear 2 can thus communicate the detected complex state ec to a third-party device 7, notably a non-smart object supervisor making it possible to trigger an intervention from the user of the object, from a technician, or a processing of a supervisor device notably to transmit a message that is a function of the complex state to a communication device of a user, of a technician and/or correct the malfunction when the complex state is an operating anomaly.

The sound s emitted by the object 4 is picked up either by a sensor 10 external to the electronic ear 2, or a sensor 210 implemented in the electronic ear 2. A sound is understood to be not only the sounds that are audible to a human being but also all other vibrations perceived by a living being or a machine, therefore also infrasound, ultrasound, but also mechanical vibration, etc.

The sound picked up sc is supplied to the first analyzer 21 capable of recognizing sounds, that is to say of identifying the movement, the friction, etc. of the object 4 originating the sound (closure of a door, placement of a key on a table, noise from an electric motor, etc.). Possibly, the sensor 10, 210 and the first analyzer 21 are implemented in one and the same sound processing device (not illustrated) which is a processing device implemented in the electronic ear 2.

In particular, the first analyzer 21 performs a sound recognition as a function of the context cx in which the sound s is emitted and/or picked up. Thus, the sound recognition takes account of the environment in which the sound is emitted and/or picked up: such as the position of the electronic ear 2 with respect to the object 4 (orientation, distance, movement, etc.), the type of environment (volume of the room, latent sound level, etc.), etc. In particular, the first analyzer 21 comprises a filter (not illustrated) capable of eliminating an environmental noise as a function of the context in which the sound s is emitted and/or picked up. For example, the filter of the first analyzer 21 is capable of totally or partly eliminating the noise linked to a movement of the object 4 and/or of the electronic ear 2.

In particular, a third analyzer 6, 26 called context analyzer supplies, to the second analyzer 22 called complex state analyzer, the context cx in which the sound sr was recognized. The context analyzer 6, 26 is either an analyzer 6 external to the electronic ear 2, or an analyzer 26 implemented in the electronic ear 2. The context analyzer notably receives information t(s) relating to the moment of capture of the sound s for example from a clock or a time stamper of the sensor 10, 2010, information g relating to the position of the object 4 upon the emission of the sound s picked up, and other information relating to the environment of the object 4, in particular information relating to the sound environment se of the object 4 upon the capture of the sound s emitted (supplied by the sensor 10, 2010 or one of other sensors present in the environment E of the object 4) or information relating to the preceding states ep of the object 4 (simple and/or complex states ec).

FIG. 3 illustrates a simplified diagram of the method for detecting complex states according to the invention. The method for detecting complex states relating to an object ST_DT comprises a diagnosis DG determining a complex state ec relating to the object as a function of a recognition S_RCG of at least one sound s emitted by the object O. Thus, the recognized sound sr allows the detection method ST_DT to determine the complex state ec of an object O, that is to say, notably, a state of abnormal operation of the object O or a state of the object O which does not relate to its operation: for example its position on a surface, in a volume, the identity of the person having manipulated the object, etc.

In particular, the complex state ec of the object O is a complex state from among the following: a state of anomalous operation of the object, a relative position of the object, an identity of the manipulator of the object. Thus, the detection method ST_DT makes it possible to warn of a malfunction of the object O even if the object O is not smart, and/or inform of a passive parameter relating to an object O even if it is not smart, even for passive objects O: position within a closed or open volume (room, apartment, home, garden, etc.), identity of the manipulator, etc. A passive parameter is a parameter that is different from an active parameter or activity parameter, that is to say a parameter relating to the operation of the object O.

In particular, the diagnosis DG is capable of determining the complex state ec of the object O as a function of the context cx of recognition S_RCG of the sound s emitted by the object O. Thus, the diagnosis DG is capable of distinguishing two complex states corresponding to one and the same recognized sound sr according to the context cx. For example, a noise from the dishwasher can correspond to two states of the dishwasher: normal or anomaly. In this case, the context cx will allow the diagnosis DG to determine which of the two states: normal or anomaly, is the current state of the object O. For example, in a context cx of activity of the dishwasher 4, the recognized sound sr will correspond to a normal state of operation (simple state) whereas, in a context cx of stoppage of the dishwasher 4, the same recognized sound sr will correspond to a state of anomalous operation (complex state ec).

In particular, the complex anomalous state ec is a type of anomalous operation of the object.

Thus, the detection method ST_DT makes it possible not only to warn of a malfunction but, in addition, of the type of malfunction allowing a user (notably a user of the object O) or a supervisor device DD to correct, possibly, the malfunction of the object O.

In particular, the diagnosis DG receives one or more recognized sounds sr₁ . . . srₙ, from the object O, and uses at least one of the recognized sounds to determine the complex state of the object O.

In particular, the diagnosis DG using several recognized sounds sr₁ . . . srₙ from an object O to determine a complex state ec comprises a step of merging of the recognized sounds SR_FU followed by a step of modeling SR_MD of the merged sound signal that makes it possible to determine, from a database of states ST_BDD, the complex state ec corresponding to the merged sound signal sf.

The sound s emitted by the object O is picked up upon a capture of sound S_CPT implemented either by a method external to the detection method ST_DT, notably a sound recognition method S_RCG, or by the method for detecting ST_DT complex states. A sound is understood to be not only the sounds that are audible to a human being but also all other vibrations perceived by a living being or by a machine, therefore also infrasound, ultrasound, but also mechanical vibration, etc.

The sound picked up sc is supplied to a sound recognition S_RCG capable of recognizing sounds, that is to say identifying the movement, the friction, etc. of the object O originating the sound (closure of a door, placement of a key on a table, noise from an electric motor, etc.). This sound recognition S_RCG is implemented either by a method external to the detection method ST_DT, notably a sound recognition method S_RCG, or by the method for detecting ST_DT complex states. Possibly, the sound capture S_CPT and the sound recognition S_RCG are implemented by one and the same sound processing method which is either a processing method external to the detection method ST_DT, or a processing method implemented by the detection method ST_DT.

In particular, an analysis of the context CX_NZ (not illustrated in FIG. 4) supplies the diagnosis DG determining a complex state ec with the context cx in which the sound sr was recognized. The analysis of the context CX_NZ is either an analysis external to the detection method ST_DT, or an analysis implemented by the detection method ST_DT. The context analysis CX_NZ receives notably information t(s) relating to the moment of capture of the sound s, for example of a time stamping H (not illustrated) of the capture of the sound S_CPT, information g relating to the position of the object O upon the emission of the sound s picked up, and other information relating to the environment of the object O, in particular information relating to the sound environment se of the object O upon the capture of the sound s emitted (supplied by the sound capture S_CPT or another capture SE_CPT relating to the environment E of the object O (not illustrated)) or information relating to the preceding states ep of the object O (simple and/or complex states ec).

In particular, the detection method ST_DT comprises a transmission EC_TR (not illustrated) suitable for transmitting, notably the diagnosed complex state, to a third-party device 7. The transmission EC_TR can transmit either the detected complex state ec, or a message comprising or that is a function of the detected complex state mssg(ec), etc. Thus, the third-party device DD can implement a processing as a function of the complex state ec received or simply displace/reproduce this information to the user of the third-party device 7.

FIG. 4 illustrates a simplified diagram of the method for monitoring objects according to the invention. One subject of the invention is also a method for monitoring objects OMNT comprising a recognition S_RCG of at least one sound s₁ . . . sₙ emitted by an object O and a diagnosis DG of the complex state ec of the object O as a function of at least one recognized sound sr₁ . . . srₙ.

The method for monitoring objects OMNT comprises a sound recognition S_RCG capable of recognizing at least one sound s emitted by an object O and a diagnosis DG capable of determining the complex state ec of the object O as a function of at least one recognized sound sr. Thus, the recognized sound sr allows the method for monitoring objects OMNT to determine the complex state ec of an object O, that is to say notably a state of abnormal operation of the object O or a state of the object O which does not relate to its operation: for example its position on a surface, in a volume, the identity of the person having manipulated the object.

In particular, the complex state ec of the object O is a complex state from among the following: a state of anomaly relating to the object, a relative position of the object, an identity of the manipulator of the object, etc. Thus, the method for monitoring objects OMNT makes it possible to warn of a malfunction of the object O even if the object O is not smart, and/or inform of a passive parameter relating to an object O even if it is not smart, even for passive objects O: position in a closed or open volume (room, apartment, home, garden, etc.), identity of the manipulator, etc. A passive parameter is a parameter that is different from an active parameter or activity parameter, that is to say a parameter relating to the operation of the object O.

In particular, the complex anomaly state ec is a type of anomaly relating to the object. Thus, the method for monitoring objects OMNT makes it possible not only to warn of a malfunction but, in addition, of the type of malfunction allowing a user (notably a user of the object O) or a supervisor device DD to correct, possibly, the malfunction of the object O.

In particular, the diagnosis DG is capable of determining a complex state ec of the object O as a function of the context cx of recognition of the sound s emitted by the object O. Thus, for the same recognized sound, the diagnosed complex state ec will not be the same as a function of the context cx. For example, in the context cx of an appliance O with the door open, a sound s from the appliance O will correspond to a normal state of operation (simple state), whereas, in a context cx of the appliance O with the door closed, this same sound s will be diagnosed as corresponding to an anomaly state (complex state ec).

In particular, the method for monitoring objects OMNT comprises a step of sound capture S_CPT capable of capturing at least one sound emitted s by the object O. Thus, the errors linked to the degradation of the sound upon the transmission between the capture S_CPT and the sound recognition S_RCG are eliminated.

In particular, the diagnosis DG receives one or more recognized sounds sr₁ . . . srₙ, from the object O, and uses at least one of the recognized sounds to determine the complex state of the object O.

In particular, the sound recognition S_RCG comprises a step of classification of the sounds picked up S_CL followed by a step of modeling S_MD of the classified sound picked up s_{cc1} . . . s_{ccn} making it possible to determine, from a database of sounds SR_BDD, the recognized sound sr₁ . . . srₙ corresponding to the classified sound picked up s_{cc1} . . . s_{ccn}. In particular, the sound recognition S_RCG is performed as a function of the context cx in which the sound s is emitted and/or picked up. Thus, the sound recognition takes account of the environment in which the sound is emitted and/or picked up: such is the position of the electronic ear 2 with respect to the object 4 (orientation, distance, movement, etc.), the type of the environment (volume of the room, latent sound level, etc.), etc. Notably, the sound recognition S_RCG filters (not illustrated) an environmental noise as a function of the context in which the sound s is emitted and/or picked up. For example, this filtering can totally or partly eliminate the noise linked to a movement of the object 4 and/or of the electronic ear 2.

In particular, the diagnosis DG comprises a step of modeling SR_MD of the recognized sound signal making it possible to determine, from a database of states ST_BDD, the complex state ec corresponding to the recognized sound signal. When the diagnosis DG uses several recognized sounds sr₁ . . . srₙ from an object O to determine a complex state ec, the diagnosis DG comprises a step of merging of the recognized sounds SR_FU followed by the step of modeling SR_MD of the merged sound signal making it possible to determine, from a database of states ST_BDD, the complex state ec corresponding to the merged sound signal sf.

In particular, the method for monitoring objects OMNT comprises a supervision of objects OMGT (not illustrated in FIG. 4) comprising at least one step of determination TRT_DT of at least one processing trt to be executed as a function of the diagnosed complex state ec. Thus, the method for monitoring objects OMNT makes it possible, by virtue of the supervision of objects OMGT, not only to diagnose a complex state ec, notably an anomaly, but also to trigger an action trt as a function of this complex state: transmission EM of a message mssg that is a function of the diagnosed complex state, correction of the diagnosed anomaly either by a human being or by the supervision OMGT of the method for monitoring objects OMNT (which notably controls either the object O if it is smart, or a third-party device DD), or by a remote supervisor DD.

In particular, the method for monitoring objects OMNT comprises a transmission EM capable of establishing a communication on command cmd from the supervision method OMGT, when the processing to be executed comprises a connection with a remote device 7. The transmission step EM can notably transmit either the detected complex state ec, or a message comprising or that is a function of the detected complex state mssg(ec), etc. Thus, the third-party device DD can implement a processing as a function of the complex state ec received or simply display/reproduce this information to the user of the third-party device DD. The third-party device DD can be a simple display screen, a smartphone, a computer of the user of the object or of a third-party user (parent, technician, etc.), a server or device providing a service in an Internet network, etc. Thus, the method for monitoring objects OMNT makes it possible not only to diagnose an anomaly but also to trigger the remote management thereof: by a remote supervisor device, a user, a technician, etc.

Notably, the monitoring method OMNT comprises a method for detecting ST_DT complexes relating to an object O implementing a diagnosis DG capable of determining a complex state ec relating to the object as a function of a recognition S_RCG of at least one sound s emitted by the object O.

Furthermore, the method for monitoring objects OMNT comprises a communication ALT, EM of the detected complex state ec to a third-party device DD, notably a supervisor of non-smart objects making it possible to trigger an intervention from the user of the object, from a technician, or a processing of a supervisor device notably to transmit a message that is a function of the complex state to a communication device of a user, of a technician and/or correct the malfunction when the complex state is an operating anomaly. In particular, the communication ALT of the diagnosed complex state ec can be performed by modification of a predetermined indicator (led, etc.), by display of a message on a screen, by the voicing of a message by means of loudspeakers, of a device implementing the monitoring method OMNT, notably an electronic ear 2.

The sound s emitted by the object O is picked up in a sound capture S_CPT implemented either by a method external to the monitoring method OMNT, or by the monitoring method OMNT. A sound is understood to mean not only the sounds that are audible to a human being but also all other vibrations perceived by a living being or a machine, therefore also infrasound, ultrasound, but also mechanical vibration, etc. The sound picked up sc is supplied to a sound recognition S_RCG capable of recognizing sounds, that is to say of identifying the movement, the friction, etc., of the object O originating the sound (closure of a door, placement of a key on a table, noise from an electric motor, etc.). Possibly, the capture S_CPT and the sound recognition S_RCG are implemented by one and the same sound processing method (not illustrated) which is a processing method implemented by the monitoring method OMNT.

In particular, a context analysis CX_NZ supplies the diagnosis DG with the context cx in which the sound sr was recognized. The context analysis CX_NZ is implemented either by an analysis method external to the monitoring method OMNT, or by the monitoring method OMNT. The context analysis CX_NZ receives notably information t(s) relating to the moment of the capture of the sound s, for example from a clock or a time stamper of the sensor 10, 2010, information g relating to the position of the object O upon the transmission of the sound s picked up, and other information relating to the environment of the object O, in particular information relating to the sound environment se of the object O upon the capture of the sound s emitted (supplied by the sensor 10, 2010 or one of other sensors present in the environment E of the object O) or information relating to the preceding states ep of the object O (simple and/or complex states ec). In particular, the context analysis CX_NZ receives at least one signal, notably sound, called environmental signals se, emitted by the environment E of the object O and performs a recognition of these environmental signals SE_RCG. Thus, the context cx supplied to the diagnosis DG by the context analysis CX_NZ will include the recognized signal ser. In particular, when the context analysis CX_NZ has several recognized environmental signals ser or not, such as the position g of the object O, the time stamping tᵢ(ₛᵢ) of the ith sound picked up emitted by the object O, etc., the context analysis CX_NZ notably comprises a merging step E_FU supplying the context cx.

FIG. 5 illustrates a simplified diagram of the method for monitoring objects according to the invention. One subject of the invention is also a method for supervising objects OMGT comprising a determination of a processing to be executed TRT_DT as a function of the complex state ec diagnosed as a function of a recognition SRCG of at least one sound s emitted by the object O. The determination of the processing to be executed provides either an identifier of a processing to be executed trt id, or the processing to be executed trt, notably in the form of a series of steps of a processing method to be implemented or instructions of a program that is executable notably by a processor.

In particular, the supervision method comprises a generation GN of a processing as a function of the diagnosed complex state ec of the object O.

In particular, the supervision method OMGT comprises a triggering of the execution of the determined processing TRT_X.

The generation of the processing GN is either implemented by the determination of the processing TRT_DT (not illustrated), or following the determination of processing TRT_DT, or by the triggering of execution of a processing TRT_X (illustrated by FIG. 5). Notably, the determination of processing controls the generation GN of a specific processing, notably by supplying the identifier of the processing to be generated trt id.

In particular, the supervision method OMGT checks I ? whether the processing is to be executed by the supervision method. If such is the case [Y], the supervision method comprises a step of execution of the determined processing trt (notably generated GN or recovered (not illustrated) from a database of processings (not illustrated) by the supervision method OMGT or the determination of processing TRT_DT, etc.). The verification I ? triggers x_trg the execution XI of the processing trt by the supervision method OMGT, notably by the electronic ear 2 implementing the supervision method OMGT. Possibly, otherwise [N], the supervision method comprises a step of transmission of the triggering of the execution of the determined processing trt (notably generated GN or recovered (not illustrated) from a database of processings (not illustrated) by the supervision method OMGT or the determination of processing TRT_DT, etc.). The verification I ? triggers em_trg the transmission EM, by the supervision method OMGT, of a command, of a message comprising either the identifier of the determined processing, or the determined processing trt, or an address for recovery of the determined processing, to either a remote device DD.

In a particular embodiment, the supervision method OMGT is implemented by the electronic ear 2 implementing the diagnosis DG of complex state ec of the object O. In this case, the monitoring method OMGT can also comprise the diagnosis DG, and possibly the sound recognition S_RCG and/or the context analysis CX_NZ, as described in relation to FIGS. 3 and 4.

In a particular embodiment, the supervision method OMGT is implemented by a third-party device distinct from the electronic ear 2, notably a smartphone, a tablet, a computer or a supervisor of detectors of complex states 20 and/or of electronic ears 2 of objects O. The detector of complex states 20 implements the detection method ST_DT described by FIG. 3 or the electronic ear 2 implements the monitoring method OMNT described by FIG. 4 which dialogues with the supervision method to which it (the detection method ST_DT or the monitoring method OMNT) supplies the diagnosed complex state ec.

A particular embodiment of at least one method according to the invention: detection method ST_DT, and/or monitoring method OMNT, and/or supervision method OMGT, is a program comprising program code instructions for executing the steps of the method for detecting anomalies and/or the method for monitoring objects and/or the supervision method when said program is run by a processor.

On the one hand, solutions are being developed to exploit vision (image, video) and recognize objects in these visual contents. On the other hand, many solutions exist for recognizing sounds, but rather in the framework of media (recognition of music, recognition of a jingle), of incidents (break glass, fire alarms), or of speech (“speech to text”, “wake-up words” technologies). Few solutions based on sound make it possible to identify objects, their state or the events associated with these objects, technically vision-based solutions (cameras) have to be involved. The only solutions which emerge are hyper-specialized, are limited to sound events that are lengthy and rather linked to safety (of the home), not the detection of an object and of its state. Therefore currently, if I do not want to use a visual capture system, I cannot detect an object (even less so if it is not connected), or its state. Furthermore, if a user wants to know the specific state of a manufactured object having a motor, he or she must either take it to an expert, or call out an expert. Even if he or she notices that the emitted sound is different from the usual sound, he or she has no help in understanding this change. The invention proposes, notably to address this lack, a detector of complex states of objects comprising an analyzer capable of determining a complex state relating to the object as a function of a recognition of at least one sound emitted by the object.

FIGS. 6a and 6b illustrate diagrams of a case of use of the invention: respectively a detector or an electronic ear that can be fixed onto an object, said detector or ear positioned on a refrigerator constituting the object. The detector 20 or the electronic ear 2 comprises at least one sensor 210 notably consisting of a microphone, notably directional in order to limit the capture of sound not originating from the object and therefore constituting a noise.

In particular, the detector 20 or the electronic ear 2 comprises fixing agents, such as magnets 28a that allow the electronic ear 2 to be fixed onto an object 4 comprising at least one metallic outer surface; suckers 28b allow for a fixing onto an object having a non-metallic flat outer surface, etc.

In particular, the detector 20 or the electronic ear 2 comprises at least one indicator lamp 27, such as an LED, making it possible to display a predetermined complex state ec notably a given indicator lamp corresponding to a predetermined complex state (the lighting of the lamp meaning that the object is then in the corresponding complex state) or an indicator lamp that can take several colors: a color of the lamp is associated with a predetermined complex state, etc.

In particular, the detector 20 or the electronic ear 2 comprises at least one socket 29, notably a power supply socket and/or a socket that can be used to connect a cable for a data link to a smartphone, a tablet, etc.

Possibly, the detector 20 or the electronic ear 2 comprises an activation button (or on/off button) making it possible to manage the energy consumption of the detector 20 or of the electronic ear 2 and also limit the periods of time during which the diagnosis of complex states is performed (no diagnosis at night or for certain objects when the user is not in the room or the home where the object is located, etc.).

By virtue of the magnets 28a and/or the sucker 28b, the detector 20 or the electronic ear 20 is positioned on the object 4, in our example the refrigerator. That notably makes it possible to devote the diagnosis implemented by the electronic ear 2 to the complex states of this object 4: the refrigerator.

The electronic ear 2 illustrated by FIGS. 6a and 6b that can be positioned on the object 4 is also called “ear patch” or “golden ear patch” referring to the submariners detecting submerged entities based on the sound produced by their movement in the water. This is a solution which aims to assist the user in identifying and having a level of clarification concerning an “abnormal” operation or operation that is different from a “normal” state of an object 4.

The electronic ear 2 is composed, for example, of a physical object (the patch) which will take measurements (picked up the sounds, possibly recognize them) and an algorithm which will analyze these measurements to calculate therefrom a probability of deviation from a “normal” state model (or toward another state): that is to say, an algorithm for diagnosing complex states. The patch notably comprises a sensor, such as a microphone 210, which picks up and/or records the sounds and the vibrations of the appliance 4 (the analysis of the vibrations can make it possible to either identify these vibrations as context [e.g. the sound when the door of the object is closed] or as the element to be analyzed [which is an input like the audible sound]). The information deriving from the sounds, vibrations picked up are compared to models and thus make it possible to identify the correct operation or the risk of malfunction of the object upon a diagnosis. These models can possibly take account of the context, notably the time and the day of the sound events/vibrations. Possibly, a user can position the electronic ear 2 on a first object O whose complex state is to be checked, in this case the refrigerator. The electronic ear 2 detecting a complex anomaly state will possibly be able to indicate an anomaly by virtue of an indicator, or send a message to a communication terminal of the user with an identifier of the anomaly, or trigger a search for the page of the user manual of the object relating to the detected anomaly to send it to and/or display it on a communication terminal of the user (tablet, smartphone, television, etc.), or trigger a connection with an assistance platform (human assistance and/or artificial intelligence) specific to the anomaly (thus the user does not waste time explaining the problem), or trigger an order for parts to be replaced as a function of the detected anomaly and/or an appointment with a technician. Once, the user to be finished the management of the refrigerator, he or she can switch off the electronic ear pending a subsequent use.

In particular, the electronic ear 2 is not dedicated to a specific object (in this case the refrigerator) because its analyzers (first and/or second) allow it to detect complex states of several distinct objects. Thus, the user who has finished managing the refrigerator can, at another time (that is to say immediately or subsequently), position the electronic ear 2 on another appliance (not illustrated) to be managed.

FIG. 6c illustrates a multi-object detector or electronic ear. The electronic ear 2 is in this case placed in a room (for example on a table) to diagnose the complex states of several objects: the table O4, the hot plate O2, the oven O3, the extractor O1, etc., which emit sounds: respectively the sounds s4, s2, s3, s11 and s12. Thus, the electronic ear 2 illustrated by FIG. 6c does not monitor the objects on which it is placed, but detects the objects O1, O2, O3, etc. in its environment and/or an object O5 which comes to interact with the object O4 on which the patch is present.

In the case where the electronic ear 2 detects the interaction of an object O5 with the object O4 on which it is present, it is also called table patch—golden ear or electronic table ear. It then addresses different problems, depending on the reference models that are being used:
Monitoring of object O5 placed on or interacting with the object/surface O4 on which the patch 2 is deposited. Typically, when the object to be monitored cannot have a patch deposited on it (for example a non-solid material: fluids; example of small objects or objects of overly complex design: toys, mechanical parts, etc.; example of moving objects on a surface).
Detecting objects O5 or events concerning objects without using vision technologies (camera)

Just like the electronic ear 2 of FIGS. 6a and 6b, the electronic ear 2 of FIG. 6c notably comprises a physical device and implements a complex state detection algorithm. The physical device notably comprises a sensor of audio signals and/or of vibrations, and/or to be time stamped, that is to say time stamp information associated therein with the signal picked up (time stamp being typically in the form (hour/min and day)). The electronic ear makes it possible to identify the objects O5 placed or moved on the cabinet or the surface O4 (for example of a table, a wall pocket, a bedside table, a parquet floor). Thus, the electronic ear 2 can recognize the arrival of a new object (bundle of keys deposited on a table, mail deposited on a table, a plate deposited on a table, etc.) or an event of an object (bundle of keys that is picked up, stack of mail that is moved, etc.).

In the case where the electronic ear 2 detects the objects O1, O2, O3 within its listening range, it is also called remote patch—golden ear or remote electronic ear. This electronic ear 2 is a “golden ear” patch as illustrated in FIGS. 6a and 6b but not positioned on an object listened to O1, O2, O3. The electronic ear 2 picks up the audio signals and/or vibrations, and/or determines the time stamping of signals picked up (hour/min and day). It makes it possible to identify the objects O1, O2, O3 within its listening range (noise of a refrigerator, noise of the alarm of a washing machine, beep from a hot plate O2, etc.). It makes it possible to detect the events of these objects (hot plate switched on O2, washing machine which has finished its cycle, etc.) or the state of an object (refrigerator which starts humming, mains adaptor which begins to make a noise, etc.).

This remote electronic ear 2 is either a standalone object, or a functionality of an existing object (for example a smart enclosure like Orange Djingo, Google Home (registered trademarks), etc.) or software on an appliance of computer or smartphone type. It can thus be embedded in a connected enclosure or on a ceiling mount (for example: light fitting or smoke detector, etc.).

The same electronic ear 2 can also have two, remote electronic ear and/or table electronic ear functions.

In the case of the electronic ear, the embedding of it in another appliance, notably a roaming appliance such as a smartphone, a connected enclosure, etc., had been considered. Thus, the electronic ear can be movable, that is to say listen to and diagnose the complex states of at least one object while the electronic ear is moving around. This mobile electronic ear, also called “mobile golden ear” thus makes it possible to move a “golden ear remote patch” as illustrated in FIG. 6c to a “listening” (observation) zone. It can then listen when it is moving or when it is immobile. These are two listening modes which are different, the listening while moving will not be able to detect the same types of information as when it is static, because of the noise associated with the movement. Notably, the mobile electronic ear mobile comprises a first analyzer specifically for listening for certain sound/noise models suited to the listening mode notably when moving. For example, the first analyzer of a mobile electronic ear will comprise a filter (of all or just a part) of the noise linked to the movement of the electronic ear. The movement of the electronic ear can be done on land (wheels, tracks), in the air (drone), on a cable, on water (float, dinghy, boat, etc.). It will be either autonomous (according to a preestablished plan, according to an autonomous movement algorithm), or remotely guided (whether the golden ear mobile is in the line of sight of the pilot, or remotely via a remote communication system), or guided by a mechanical system (for example a cable), or unguided (drift on a body of water)

The invention also targets a medium. The information medium can be any entity or device capable of storing the program. For example, the medium can comprise a storage means, such as a ROM, for example a CD ROM or a microelectronic circuit ROM, or even a magnetic storage means, for example a diskette or a hard disk.

Also, the information medium can be a transmissible medium such as an electrical or optical signal which can be routed via an electrical or optical cable, wirelessly or by other means. The program according to the invention can in particular be downloaded over a network, notably of Internet type.

Alternatively, the information medium can be an integrated circuit in which the program is incorporated, the circuit being adapted to execute or to be used in the execution of the method concerned.

In another implementation, the invention is implemented by means of software and/or hardware components. In that respect, the term module can correspond equally to a software component or to a hardware component. A software component corresponds to one or more computer programs, one or more subroutines of a program, or more generally to any element of a program or of software capable of implementing a function or a set of functions according to the above description. A hardware component corresponds to any element of a hardware assembly capable of implementing a function or a set of functions.

1. A detector configured to detect complex states of objects, the detector comprising:
an analyzer configured to determine a complex state relating to the object as a function of a recognition of at least one sound emitted by the object.

2. The detector of claim 1, wherein the complex state of the object is a complex state from among the following: a state of anomalous operation of the object, a relative position of the object, an identity of the manipulator of the object.

3. The detector of claim 1, wherein the analyzer is a complex state of the object as a function of the context of recognition of the sound emitted by the object.

4. The detector of claim 2, wherein the complex state is a complex anomalous state, and wherein the complex anomalous state is a type of anomalous operation of the object.

5. The detector of claim 1, wherein the detector is a connected detector.

6. An electronic ear comprising:
a first analyzer configured to recognize at least one sound emitted by an object; and
a second analyzer configured to diagnose a complex state of the object as a function of the at least one recognized sound.

7. The electronic ear of claim 6, wherein the electronic ear comprises a sound sensor configured to capture at least one sound emitted by the object.

8. The electronic ear of claim 6, wherein the electronic ear comprises an object supervisor configured to determine at least one processing to be executed as a function of the diagnosed complex state.

9. The electronic ear of claim 8, wherein the electronic ear comprises a communication interface configured to establish a communication on command from the supervisor, when the processing to be executed comprises a connection with a remote device.

10. The electronic ear of claim 9, wherein, when the electronic ear comprises fixing agents and is intended to be fixed onto the object emitting the recognized sound, the sensor is positioned in the part of the electronic ear close to the fixing agents and/or oriented in the electronic ear toward the plane formed by the fixing agents.

11. A method for detecting complex states relating to an object, the method comprising:
performing a diagnosis, the diagnosis determining a complex state relating to the object as a function of a recognition of at least one sound emitted by the object.

12. The method of claim 11, further comprising recognizing at least one sound emitted by an object, wherein the diagnosis determining the complex state of the object is a function of at least one recognized sound.

13. The method of claim 11, further comprising determining a processing to be executed as a function of the complex state diagnosed as a function of a recognition of at least one sound emitted by the object.

14. The method of claim 13, wherein the supervision method comprises triggering the execution of the determined processing.

15. A non-transitory, computer readable storage medium having stored thereon instructions which, when executed by a processor, causes the processor to perform the method of claim 11.
